SEQUENCE LISTING (12 sequences)

SEQ ID NO: 1 - DNA, 5193 bp, Artificial Sequence
  pS-PACAP38
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtgacgcggcccagccggccaggcgcaggaggcactcggacgggatcttcacggacagctacagccgctaccggaaacaaatggctgtcaagaaatacttggcggccgtcctagggaagaggtataaacaaagggttaaaaacaaaggatgacgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 5190 bp, Artificial Sequence
  pS-CGRP
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtgacgcggcccagccggccaggcgcaggaggtcctgcaacactgccacctgtgtgacccatcggctggcaggtctgctgagcagatcaggaggtgtggtgaaggacaactttgttcccaccaatgtgggctctgaagccttcggctgacgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 30 bp, Artificial Sequence
  primer
cgcgaattcaccatggccggacgcgcccgc

SEQ ID NO: 4 - DNA, 27 bp, Artificial Sequence
  primer
ggctctagatcaggaggaaggtgcagg

SEQ ID NO: 5 - DNA, 34 bp, Artificial Sequence
  primer
cgcaagctttcactcggacgggatcttcacggac

SEQ ID NO: 6 - DNA, 32 bp, Artificial Sequence
  primer
gcggcggccgctcatcctttgtttttaaccct

SEQ ID NO: 7 - DNA, 31 bp, Artificial Sequence
  primer
cgcgaattcaccatggagacagacacactcc

SEQ ID NO: 8 - DNA, 31 bp, Artificial Sequence
  primer
cggcgtctagatcatcctttgtttttaaccc

SEQ ID NO: 9 - DNA, 30 bp, Artificial Sequence
  primer
cgcaagcttttcctgcaacactgccacctg

SEQ ID NO: 10 - DNA, 35 bp, Artificial Sequence
  primer
gtgggctctgaagccttcggctgagcggccgccgc

SEQ ID NO: 11 - DNA, 28 bp, Artificial Sequence
  primer
cgcgaattcaccatggagacagacacac

SEQ ID NO: 12 - DNA, 30 bp, Artificial Sequence
  primer
gtgggctctgaagccttcggctctagacgc

(19) United States
(12) Patent Application Publication
(10) Pub. No.: US 2012/0058950 A1
(43) Pub. Date: Mar. 8, 2012

(54) DETECTION OF NEUROPEPTIDES ASSOCIATED WITH PELVIC PAIN DISORDERS AND USES THEREOF

(75) Inventors: Ronald W. Wood, Rochester, NY (US); Jay Reeder, East Rochester, NY (US); Edward M. Schwarz, Rochester, NY (US); Edward M. Messing, Rochester, NY (US); Susan R. Schoen, Macedon, NY (US); Margaret A. Vizzard, South Burlington, VT (US); Ian Dickerson, Pittsford, NY (US)

(73) Assignees: University of Vermont and State Agricultural College, Burlington, VT (US); UNIVERSITY OF ROCHESTER, Rochester, NY (US)

(21) Appl. No.: 13/295,036

(22) Filed: Nov. 11, 2011

Related U.S. Application Data
(63) Continuation of application No. 10/577,395, filed on Apr. 27, 2007, now abandoned.
(60) Provisional application No. 60/515,408, filed on Oct. 29, 2003.

(51) Int. Cl. A61K 38/02; A61P 29/00; A61K 31/517; G01N 33/566; A61K 31/426

(52) U.S. Cl. 514/18.3; 435/7.92; 436/501; 514/369; 514/252.17

(57) ABSTRACT

Diagnostic assessment and therapeutic treatment of pelvic pain disorders, including bladder disorders, bowel disorders, and/or reproductive tissue or organ disorders that are characterized by increased expression of the neuropeptides CGRP and/or PACAP. Additionally, applicants have developed a transgenic non-human model for pelvic pain disorders, where the transgenic animal expresses in bladder sensory neurons a recombinant neuropeptide implicated in the pelvic pain disorder.

This application is a continuation of U.S. patent application Ser. No. 10/577,395, which is a national application under 35 U.S.C. 371 of PCT Application No. PCT/US2004/036015, filed Oct. 29, 2004, and claims the priority benefit of provisional U.S. patent application Ser. No. 60/515,408, filed Oct. 29, 2003, which is hereby incorporated by reference in its entirety.

The present invention was made with funding received from the National Institutes of Health under grant DK 057679. The U.S. government has certain rights in this invention.

### FIELD OF THE INVENTION

The present invention relates to detecting levels of neuropeptides associated with pelvic pain disorders, including bladder disorders such as interstitial cystitis, and uses thereof.

### BACKGROUND OF THE INVENTION

Interstitial cystitis is a chronic pelvic-perineal pain syndrome of unknown etiology. The clinical features of interstitial cystitis include chronic recurrent urinary frequency, urgency, and pain referable to the lower urinary tract. These symptoms often appear acutely and generally follow a waxing and waning course (Sant, Interstitial Cystitis, Lippincott-Raven (1997); Hanno, “Interstitial Cystitis and Related Diseases,” In: Cambell's Urology, 7^{th} ed., Walsh et al. (Eds.), p. 631 (1998). Epidemiological studies reveal that more than half of the patients with interstitial cystitis report daily or constant pain, which is exacerbated by stressful circumstances (Koxiol, Urol Clin North Am 21:7 (1994)).

A set of inclusion and exclusion criteria for the diagnosis of interstitial cystitis has been established by the National Institutes of Arthritis, Diabetes, Digestive and Kidney Diseases to ensure that subjects in research studies of interstitial cystitis are reasonably comparable (Gillenwater and Wein, J Urol 140:203 (1998)). These criteria are more stringent than those often used by practicing urologists (Hanno et al., J Urol 161:553 (1999)). The diagnosis usually includes the presence of compatible clinical features and the absence of objective evidence of other diseases that may cause the symptoms. At least 2 variants of cystoscopy findings have been described in patients with interstitial cystitis (Johansson and Fall, J Urol 140:143:1118 (1990)). In the more common form, only glomerulations (punctate submucosal petechial hemorrhages) are seen at cystoscopy. In the less common ulcer form first described in 1918 by Hunner (JAMA 70:203 (1918)), fissures and scars that crack and bleed when the bladder is distended are present.

When interstitial cystitis is suspected in a patient, histopathological studies may be done to rule out other, better defined possible diagnoses, but findings in the bladder of patients with interstitial cystitis are inconsistent. In the nonulcer form of the disease, scattered glomerulations, small mucosal tears, and submucosal hemorrhages with no or a mild inflammatory infiltrate can be seen. Abnormalities are usually limited to vasodilatation and submucosal edema (Hanno, “Interstitial Cystitis and Related Diseases,” In: Campbell's Urology, 7^{th} ed., Walsh et al. (Eds.), p. 631 (1998). In the classic form, the suburothelium shows chronic inflammation, fibrosis, dilatation of vessels with hemorrhage, neural proliferations and perineuritis (Tomaszewski et al., Urology 57:67 (2001)). These abnormalities occurred in only 3.9% of the 209 biopsy samples in the interstitial cystitis database study. Tomaszewski et al. also reported that histopathological lesions in patients with interstitial cystitis are not unique to interstitial cystitis, nor did lesion severity correspond well with nighttime urinary frequency, urgency and/or pain (Tomaszewski et al., Urology 57:67 (2001)). Moreover, no reliable correlation has been found of the severity of cystoscopy findings with the clinical symptoms of interstitial cystitis (Denson et al., J Urol 164:1908 (2000)), and the presence of glomerulations is not even restricted to interstitial cystitis (Waxman et al., J Urol 160:1663 (1998)).

The understanding of interstitial cystitis is further complicated by the observations that symptoms may remain even after removal of the bladder (Baskin and Tanagho, J Urol 147:683 (1992)), and bladder lesions can be present in patients reporting significant improvement in clinical signs (Thilagarajah et al., BJU Int 87:207 (2001)). Patients also appear to have various problems not related to bladder function. For example, epidemiological studies of patients with interstitial cystitis have also noted that other nonbladder related symptoms, such as headache, cough, and tingling in fingers and/or toes can be more prevalent in these patients than in age matched controls. Those with interstitial cystitis also report other chronic pain conditions, such as migraine (Barkhuizen, Curr Pain Headache Rep 5:351 (2001)), fibromyalgia (Clauw et al., J Psychiatr Res 31:125 (1997)), and the irritable bowel syndrome (Alagiri et al., Urology 49:52 (1997)). These observations suggest that the abnormalities responsible for interstitial cystitis may extend beyond the bladder.

The present invention is directed to overcoming these limitations in the diagnosis and treatment of pelvic pain disorders generally, and more specifically with regard to the disorder known as interstitial cystitis.

### SUMMARY OF THE INVENTION

A first aspect of the present invention relates to a method of diagnosing a pelvic pain disorder that includes the steps of measuring a level of Calcitonin Gene-Related Peptide (CGRP) or Pituitary Adenylate Cyclase Activating Peptide (PACAP), or both, in a patient sample; and determining if the measured level of CGRP or PACAP, or both, in the patient sample is elevated in relation to a standard level of CGRP or PACAP in a normal asymptomatic population, wherein the measured level of CGRP or PACAP, or both, that is elevated relative to the standard level indicates the diagnosis of a pelvic pain disorder.

A second aspect of the present invention relates to a method of determining predisposition of an individual to conditions associated with pelvic pain disorders that includes the steps of measuring a level of CGRP or PACAP, or both, in a sample obtained from an individual; and determining if the measured level of CGRP or PACAP, or both, in the sample is elevated in relation to a standard level of CGRP or PACAP in a normal asymptomatic population, wherein the measured level of CGRP or PACAP, or both, that is elevated relative to the standard level indicates the individual is predisposed to conditions associated with a pelvic pain disorder.

A third aspect of the present invention relates to a method of treating a pelvic pain disorder in a patient that includes the steps of providing a CGRP antagonist; and administering the CGRP antagonist to a patient in an amount effective to treat the pelvic pain disorder. According to this aspect of the invention, treatment of the pelvic pain disorder can be effective to mitigate symptoms of the pelvic pain disorder.

A fourth aspect of the present invention relates to a method of characterizing response to treatment for a pelvic pain disorder that includes the steps of measuring a level of CGRP or PACAP, or both, in a sample obtained from a patient to be treated for a pelvic pain disorder; treating the patient with a CGRP or PACAP antagonist; and repeating said measuring after said treating, whereby a decrease in the CGRP or PACAP level, or both, following said treating indicates that the treatment is effective.

A fifth aspect of the present invention relates to a transgenic non-human mammal that includes a first DNA construct that is expressed in bladder sensory neurons, the first DNA construct having a promoter operatively coupled to a DNA molecule encoding a neuropeptide. According to one embodiment, a bi-transgenic system is utilized to prepare a transgenic non-human mammal whose somatic and germ cells are transformed. For the bi-transgenic system, the first DNA construct includes an inducible promoter that possesses a tetracycline response element and is inducible in the presence of an rtTA protein and doxycycline, and the second DNA construct includes a promoter that is specific for urothelial tissues and a DNA molecule encoding the rtTA protein. According to another embodiment, an infective transformation system is utilized to prepare a transgenic non-human mammal having bladder sensory neurons that express neuropeptide.

A sixth aspect of the present invention relates to a recombinant CGRP or PACAP polypeptide that is amidated at its carboxyl terminus (i.e., present in an active form)

A seventh aspect of the present invention relates to one or more recombinant DNA constructs encoding the recombinant polypeptide(s) of the present invention. Related aspects include recombinant expression vectors and host cells, particularly mammalian host cells for expressing the recombinant polypeptides. The host cells can be isolated, ex vivo, or present within the transgenic organism described above, in vivo.

### BRIEF DESCRIPTION OF THE DRAWINGS

FIG. 1 is a calibration curve for an ELISA for CGRP. The curve is bounded by 95% confidence intervals.

FIG. 2 is boxplot showing a urine analysis for 9 controls and 15 patients, as measured by ELISA. Urine concentration is shown on a log scale. Each box encompasses the first to the third quartile (25 to 75%) of the distribution; the line in the middle is the median. The medians for controls and IC patient groups were 1.21 and 3.11 ng/mg creatinine respectively. Note that 75% of the IC patients had CGRP levels greater than 1.92 ng/mg creatinine, and that 75% of control subjects had levels less than 1.82 ng/mg creatinine

FIG. 3A is a schematic diagram of the S-PACAP38 and S-CGRP expression constructs, parental vectors and PCR primers used in the cloning. Horizontal arrows indicate PCR primers in which the restriction sites are in bold typeface. The nucleotide sequences for the secretion constructs (pS-PACAP38 and pS-CGRP) are illustrated in FIGS. 4A-C and FIGS. 5A-C, respectively. FIG. 3B is a schematic diagram of the corresponding vector that is used in combination with the TRE-vectors, which contains the uroplakin II promoter (Zhang et al., Cancer Res. 62(13):3743-3750 (2002), which is hereby incorporated by reference in its entirety) upstream of the open reading frame for the Tet-On or rtTA open reading frame (Kistner et al., Proc Natl Acad Sci USA 93:10933-10938 (1996), which is hereby incorporated by reference in its entirety).

FIGS. 4A-C illustrate the nucleotide sequence of secretion plasmid pS-PACAP38 (SEQ ID NO: 1), which encodes the Ig-kappa—cleavage peptide—PACAP38 fusion protein. The open reading frame starts at nt 905.

FIGS. 5A-C illustrate the nucleotide sequence of secretion plasmid pS-CGRP (SEQ ID NO: 2), which encodes the Ig-kappa—cleavage peptide—CGRP fusion protein. The open reading frame starts at nt 905.

FIG. 6 illustrates the expression of rtTA protein in transgenic mouse tissues via immunohistochemistry of formalin-fixed and paraffin-embedded bladders from the UPII-rtTA transgenic mouse (left) and a wild type mouse (right). After antigen recovery, the anti-VP16 antibody (Clontech), recognized an epitope in the rtTA carboxy terminus, demonstrating urothelium-specific transgene expression.

FIG. 7 is an image showing sub-mucosal inflammatory infiltrate in bi-transgenic UPII-rtTA X TRE-erbB2 mouse bladder following three week exposure to doxycycline. This figure demonstrates that the bi-transgenic expression system is operable is mouse urothelial tissues.

FIG. 8 illustrates the secretion of expression products prepared using the pS-secretion constructs shown in FIG. 3A. The left panel is a phase contrast image of the pS-EGFP/pCRE-Luc transfected cells taken two days after addition of the plasmids to the culture media. The right panel is the green fluorescence image of the same field.

FIG. 9 is a graph illustrating luciferase activity assayed by measurement of luminescence after addition of 20 ul of cell lysate to 100 ul of Reporter Lysis Reagent. The results for three replicate measurements of luciferase activity for duplicate wells demonstrate that the CGRP and PACAP secretion constructs are triggering CRE-Luc activity in UMUC3 cells, implying the production of functional CGRP and PACAP peptides.

### DETAILED DESCRIPTION OF THE INVENTION

The present invention relates generally to the diagnosis and treatment of pelvic pain disorders, including bladder disorders that are characterized by increased expression of the neuropeptides CGRP and/or PACAP. Exemplary pelvic pain disorders include, without limitation, pain disorders involving the bladder (e.g., interstitial cystitis), pain disorders involving the bowel (e.g., Crohn's disease, ulcerative colitis, irritable bowel syndrome, etc.), as well as pain disorders involving the reproductive organs or tissues such as the uterus, vagina, cervix, testis, prostate, and epididymis (e.g., vulvodynia, vestibulitis, endometriosis, prostatitis, orchalgia, proctalgia). With respect to such diagnosis and treatment, samples will be taken from and therapeutics administered to individuals (e.g., patients).

As used herein, “patient” or “individual” refers to any mammal that exhibits a pelvic pain disorder that is characterized by increased expression of the neuropeptides CGRP and/or PACAP, or otherwise is symptomatic for the above pain disorders, particularly interstitial cystitis. Exemplary mammals include, without limitation, humans and other primates, cats, dogs, cows, horses, pigs, sheep, and rodents such as mice and rats.

One aspect of the present invention is directed to a method of diagnosing pelvic pain disorders. This method involves measuring a level of one or both of the neuropeptides calcitonin gene-related peptide (“CGRP”) or pituitary adenylate cyclase activating peptide (“PACAP”) in a patient sample and then determining whether the CGRP or PACAP level in the patient sample is elevated in relation to a level of CGRP or

PACAP in a normal asymptomatic population. Alternatively, the level of CGRP or PACAP in the patient sample may be correlated with a range associated with the pelvic pain disorder. An elevated CGRP or PACAP level in the patient sample indicates the diagnosis of a pelvic pain disorder, such as those described herein (including interstitial cystitis).

Suitable sample materials from the patient are preferably fluid samples, including, without limitation, blood, urine, and spinal fluid. Of these, urine is preferred.

When measuring CGRP, it is preferable that the active form of CGRP is being measured rather than the inactive form. The active form of CGRP differs from the inactive precursor proCGRP by the endoprotease cleavage of internal dibasic (KR) and tetrabasic (RRRR) sites and the carboxyl amidation of the product following cleavage at the tetrabasic site. The CGRP biosynthetic pathway is described in greater detail in Rosenblatt et al., Peptides 18(4):567-576 (1997). As used herein, CGRP refers to an active form thereof.

When measuring PACAP, it is preferable that one of the active forms of PACAP is being measured rather than an inactive form. Two active forms have been identified, PACAP-27 and PACAP-38, both of which are truncated from the inactive precursor proPACAP. As used herein, PACAP refers to one of the active forms thereof.

Measuring CGRP or PACAP levels in a patient sample can be performed with an assay system. The assay preferably utilizes an immunological detection procedure, using an antibody or binding portion thereof recognizing CGRP or PACAP. Exemplary CGRP antibodies include, without limitation, polyclonal anti-CGRP (rat) and polyclonal anti-CGRP (human), both available from Research Diagnostics, Inc. (Flanders, N.J.); and antibody BIBN4096BS which is available from Boehringer Ingelheim. Exemplary PACAP antibodies include, without limitation, polyclonal anti-PACAP (human, chicken, mouse, ovine, porcine, rat) available from Research Diagnostics, Inc. Other antibodies, both polyclonal and monoclonal can be raised against various peptides of CGRP and PACAP, whether fused to an immunogenic conjugate or not, using procedures well known in the art.

The patient sample is contacted with the antibody or binding portion thereof and any reaction which indicates that CGRP or PACAP is present in the patient sample is detected. Detection of antibody-CGRP or PACAP binding can be achieved using any of a variety of known detection procedures, such as enzyme-linked immunoabsorbent assay, radioimmunoassay, gel diffusion precipitin reaction assay, immunodiffusion assay, agglutination assay, fluorescent immunoassay, protein A immunoassay, or immunoelectrophoresis assay.

Prior to detection, the patient sample can optionally be concentrated to allow for greater sensitivity in performing such detection. The concentration of samples is described in detail in Rosenblatt et al., “Endoproteolysis at Tetrabasic Amino Acid Sites in Procalcitonin Gene-Related Peptide by Pituitary Cell Lines,” Peptides 18(4):567-576 (1997).

Alternatively, CGRP or PACAP levels in a patient sample can be measured using HPLC or mass spectrometry. The use of HPLC and mass spectrometry detection procedures are well known in the art.

A second aspect of the present invention is directed to a method of determining predisposition of an individual to conditions associated with or development of pelvic pain syndromes. This method is carried out substantially as described above, although the individual from whom the sample is taken is characterized by exhibiting few, if any, symptoms or conditions associated with pelvic pain syndromes, such as interstitial cystitis. An elevated CGRP or PACAP level in the sample indicates the individual is predisposed to conditions associated with a pelvic pain disorder. Conditions associated with pelvic pain disorders include, without limitation, pain during urination, urgency of urination, frequency of urination, ulcers of bladder mucosa, and petechial hemorrhages of bladder mucosa.

A third aspect of the present invention is directed to a method of treating a pelvic pain disorder in a patient. This method involves providing a CGRP or PACAP antagonist and administering the CGRP or PACAP antagonist to the patient in an amount effective to treat the pelvic pain disorder.

Suitable CGRP or PACAP antagonists can be any antibodies, small molecule therapeutics (e.g., pharmaceutical compounds), or derivatives of these peptides that interfere with CGRP or PACAP activity at their designated receptors. Exemplary CGRP antagonists include, without limitation, the antibodies described above and SB-(+)-273779 [N-methyl-N-(2-methylphenyl)-3-nitro-4-(2-thiazolylsulfinyl)nitrobenzanilide] (Smith Kline Beecham), and the CGRP8-37 fragment. Exemplary PACAP antagonists include, without limitation, the anti-PACAP antibodies described above.

The CGRP or PACAP antagonist can be administered alone or in combination, and each can be present, either together or individually, in the form of a pharmaceutical composition. The pharmaceutical composition includes the active ingredient(s) in combination with a pharmaceutically acceptable carrier.

The pharmaceutical compositions of the present invention are preferably in the form of a single unit dosage form that contains an amount of the one or more CGRP or PACAP antagonists effective to treat pelvic pain disorders of the type described herein. The pharmaceutical composition can also include suitable excipients, or stabilizers, and can be in solid or liquid form such as, tablets, capsules, powders, solutions, suspensions, or emulsions. Typically, the composition will contain from about 0.01 to 99 percent, preferably from about 5 to 95 percent of active compound(s), together with the carrier.

The CGRP or PACAP antagonist, when combined with a suitable carrier and any excipients or stabilizers, and whether administered alone or in the form of a composition, can be administered orally, parenterally, subcutaneously, transdermally, intravenously, intramuscularly, intraperitoneally, by intranasal instillation, by implantation, by intracavitary or intravesical instillation, intraocularly, intraarterially, intralesionally, by application to mucous membranes, such as, that of the nose, throat, and bronchial tubes (i.e., inhalation), or by intrabladder administration.

For most therapeutic purposes, the CGRP or PACAP antagonist can be administered orally as a solid or as a solution or suspension in liquid form, via injection as a solution or suspension in liquid form, or via inhalation of a nebulized solution or suspension.

The solid unit dosage forms of the CGRP or PACAP antagonist can be of a conventional type. The solid form can be a capsule, such as an ordinary gelatin type containing the CGRP or PACAP antagonist and a carrier, for example, lubricants and inert fillers such as, lactose, sucrose, or cornstarch. In another embodiment, the CGRP or PACAP antagonist is tableted with conventional tablet bases such as lactose, sucrose, or cornstarch in combination with binders like acacia or gelatin, disintegrating agents such as cornstarch, potato starch, or alginic acid, and a lubricant such as stearic acid or magnesium stearate.

For injectable dosages, solutions or suspensions of the CGRP or PACAP antagonist can be prepared in a physiologically and pharmaceutically acceptable diluent as the carrier. Such carriers include sterile liquids, such as water and oils, with or without the addition of a surfactant and other pharmaceutically and physiologically acceptable components, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

For use as aerosols, the CGRP or PACAP antagonist in solution or suspension may be packaged in a pressurized aerosol container together with suitable propellants, for example, hydrocarbon propellants like propane, butane, or isobutane with conventional adjuvants. The CGRP or PACAP antagonist of the present invention also may be administered in a non-pressurized form such as in a nebulizer or atomizer.

The CGRP or PACAP antagonist may be administered in combination with other therapeutic regimens that are known in the art, whether now known or hereafter developed.

A related aspect of the present invention concerns a method of mitigating symptoms associated with a pelvic pain disorder in a patient. This method involves treating the patient for the pelvic pain disorder as described above. Basically, when treating the underlying cause of the pelvic pain disorder, it is believed that management of symptoms can likewise be achieved. By management of symptoms, it is intended that the severity of symptoms can be maintained (i.e., worsening or advancement of symptoms is controlled) or, more preferably, the severity of symptoms can be reduced either in whole or in part.

A fourth aspect of the present invention is directed to a method of characterizing response to treatment for a pelvic pain disorder. This method involves measuring CGRP or PACAP level in a sample obtained from a patient to be treated for the pelvic pain disorder, treating the patient with a CGRP or PACAP antagonist, and measuring the CGRP or PACAP level again in a second sample obtained from the patient. Preferably, some time is allowed to pass before taking the second measurement—such as several days, weeks, or even months. Regardless of such delay, a decrease in the CGRP or PACAP level following the treatment with a CGRP or PACAP antagonist indicates that the treatment is effective.

A fifth aspect of the present invention relates to a transgenic non-human mammal that includes a first DNA construct that is expressed in bladder sensory neurons, the first DNA construct having a promoter operatively coupled to a DNA molecule encoding a neuropeptide (either PACAP or CGRP). The transgenic non-human mammals are characterized by overexpression (i.e., relative to non-transgenic mammals) of the neuropeptide. These transgenic animals are useful for the study of pelvic pain disorders and assessing the efficacy of potential therapeutic agents in the treatment thereof.

The transgene can be introduced, using the expression vector, into one or more target tissues or systemically to achieve subsequent expression of the encoded neuropeptide. The recombinant gene includes, operatively coupled to one another, an upstream promoter operable in mammalian cells, and other suitable regulatory elements (i.e., enhancer or inducer elements), a coding sequence that encodes the desired neuropeptide, and a downstream transcription termination region. Depending upon the desired method of transformation, i.e., somatic mosaic or transformation of somatic and germ cells, persons of skill in the art can readily selected suitable constitutive promoter, inducible, or tissue specific promoters to regulate transcription of the recombinant transgene. One of skill in the art can readily select and utilize such promoters, whether now known or hereafter developed. Known recombinant techniques can be utilized to prepare the recombinant gene, transfer it into the expression vector, and administer the vector to the non-human mammal. Exemplary procedures are described in Sambrook et al., Molecular Cloning: A Laboratory Manual, Second Edition, Cold Spring Harbor Press, NY (1989), which is hereby incorporated by reference in its entirety. One of skill in the art can readily modify these procedures, as desired, using known variations of the procedures described therein.

PACAP is an appropriate neuropeptide for overexpression in non-human animals because: PACAP is localized in fine nerves in the urothelium (Fahrenkrug and Hannibal, Neuroscience 83(4):1261-1272 (1998), which is hereby incorporated by reference in its entirety); PACAP upregulates in spinal cord after bladder injury (Vizzard, J. Comp. Neurol. 420(3):335-348 (2000), which is hereby incorporated by reference in its entirety); PACAP receptors have been localized by autoradiography in human bladder (Reubi, Ann. NY Acad. Sci. 921:1-25 (2000), which is hereby incorporated by reference in its entirety); PACAP has no bladder activity in vitro (i.e., does not contract detrusor, or alter detrusor contractions elicited electrically or cholinergically) (Ishizuka et al., Neuroscience 66(4):1009-1014 (1995), which is hereby incorporated by reference in its entirety); PACAP has activity when infused near the bladder (Ishizuka et al., Neuroscience 66(4):1009-1014 (1995), which is hereby incorporated by reference in its entirety); and 6) PACAP has been hypothesized to exert a regulatory influence over other neuropeptides. By inference, PACAP plays a role in sensory function, and overexpression in or near nerves innervating the urothelium is believed to result in any or all of the following: a) bladder “allodynia”; b) bladder “hyperalgesia” following painful insult; c) enhanced frequency and contractility. Therefore, generation of a non-human animal model overexpressing PACAP in the urothelium near nerves or within the nerves themselves would provide a model of urgency, frequency, and/or the pain of interstitial cystitis.

CGRP is also an appropriate neuropeptide for overexpression in non-human animals because: CGRP is localized in fine nerves in the urothelium ((Ishizuka et al., Neuroscience 66(4):1009-1014 (1995); Avelino et al., Neuroscience 109(4):787-798 (2002); Avelino and Cruz, Auton. Neurosci. 86(1-2):37-46 (2000), which is hereby incorporated by reference in its entirety); CGRP upregulates in spinal cord after bladder injury (J. Chem. Neuroanat. 21(2):125-138 (2001), which is hereby incorporated by reference in its entirety); CGRP receptors have been localized in rat bladder by autoradiography (Banasiak and Burcher, Peptides 15(2):333-339 (1994), which is hereby incorporated by reference in its entirety); specific binding of [¹²⁵I]CGRP was observed over the epithelium and weakly over submucosal arterioles, but not over smooth muscle, and the density of [¹²⁵I]CGRP binding sites on the epithelium, but not blood vessels, increased after chronic capsaicin pretreatment, suggesting receptor upregulation (Banasiak and Burcher, Peptides 15(2):333-339 (1994), which is hereby incorporated by reference in its entirety); CGRP has little effect on isometric tension of isolated human detrusor smooth muscle (Uckert et al., World J. Urol. 20(4):244-249 (2002), which is hereby incorporated by reference in its entirety); and CGRP (10 nmol/kg) administered intravenously decreased both mean amplitude of micturition contractions MAC and pressure threshold for micturition in the urethane-anesthetized hamster (but not in the rat at this dose) (Lecci et al., Auton. Neurosci. 91(1-2):37-46 (2001), which is hereby incorporated by reference in its entirety).

Relatively recent advances in molecular technologies have now made it possible to target increased expression of signaling molecules to specific organs or tissues, including the urothelium (Lin et al., Proc. Natl. Acad. Sci. USA, 92(3):679-683 (1995), which is hereby incorporated by reference in its entirety). By using inducible promoters, it is also possible to activate the expression of these molecules only in adult animals by making the expression dependent on the presence of an exogenously administered innocuous chemical not found in the diet or environment of the untreated animal, for example doxycycline (Saam and Gordon, J. Biol. Chem. 274(53):38071-38082 (1999), which is hereby incorporated by reference in its entirety).

One approach involves a bi-transgenic model whose somatic and germ cell lines contain the two transgenes. One transgene encodes the neuropeptide under control of an inducible promoter. A preferred inducible promoter is one that contains a tetracycline response element and is inducible in the presence of reverse tetracycline transactivator (also known as rtTA or Tet-On) and doxycycline or equivalents thereof. Other inducible promoters can also be utilized. The second transgene is a tissue specific transgene that encodes rtTA under control of a promoter that is operable only in the target tissue or organ, in this case urothelial tissues such as bladder sensory neurons. One exemplary promoter is the uroplakin II promoter (Sun et al., Mol. Biol. Rep. 23(1):3-11 (1996); U.S. Pat. No. 5,824,543 to Sun; Lin et al., Proc. Natl. Acad. Sci. USA, 92(3):679-683 (1995); Zhang et al., Cancer Res. 62(13):3743-3750 (2002), each of which is hereby incorporated by reference in its entirety), although other promoters can also be utilized.

To ensure that PACAP and/or CGRP are appropriately processed post-transcriptionally, that is, truncated to their active forms and amidated at their carboxy terminal ends, the transgenic organism can also contain a third transgene encoding peptidyl glycine α-amidating monooxygenase (PAM) under control of an inducible promoter (which can be the same or different from the inducible promoter of the transgene encoding the neuropeptide). Alternatively, the first and third transgenes can be combined together into a double recombinant transgene (i.e., expressing both the neuropeptide and PAM).

The preparation of transgenic whole organism, whose somatic and germ cells possess the transgene, can be performed according to known procedures. Basically, singly transgenic organisms can be prepared by microinjecting transgenes into fertilized eggs which are then implanted into foster mothers. After confirming that individuals possess the transgene, the positive founder animals can be backcrossed with F1 hybrids to generate hemizygous animals. Development of stable, multiply transgenic lines involves crossing of the singly transgenic lines. The resulting generation can be phenotypically and genotypically assessed to identify those individuals possessing the bi-transgene or tri-transgene phenotypes/genotypes.

Another approach involves a somatic mosaic transgenic non-human animal some of whose somatic cells, but not germ cells, have been transformed with a transgene expressing the neuropeptide. According to this approach, the transgene includes either a constitutive promoter or tissue-specific promoter, and delivery of the transgene is preferably targeted to a desired tissue, in this case urothelial tissues. According to this approach, the transgene can be prepared using standard recombinant techniques such as those described above.

For delivery of the transgene to the targeted tissues, either naked plasmid or infective transformation vectors can be employed.

Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. Adeno-associated viral gene delivery vehicles can be constructed and used to deliver a gene to cells. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l. Acad. Sci. USA 90:10613-10617 (1993); and Kaplitt et al., Nature Genet. 8:148-153 (1994), each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; and U.S. Pat. No. 5,885,808 to Spooner et al.; and U.S. Pat. No. 5,871,727 to Curiel, each of which is hereby incorporated by reference in its entirety).

Retroviral vectors which have been modified to form infective transformation systems can also be used to deliver nucleic acid encoding a desired protein or polypeptide or RNA product into a target cell. One such type of retroviral vector is disclosed in U.S. Pat. No. 5,849,586 to Kriegler et al., which is hereby incorporated by reference in its entirety. Another type of retroviral vector which is preferred for use with rodents are modified feline immunodeficiency virus vectors.

Liposomal delivery systems can be used to deliver expression vectors or plasmid DNA into targeted cells. Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of nucleic acid delivery via liposomes require that the liposome carrier ultimately become permeable and release the encapsulated contents at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive release, active release of the contents involves using an agent to induce a permeability change in the liposome vesicle. Liposome membranes can be constructed so that they become destabilized when the environment becomes acidic near the liposome membrane (see, e.g., Proc. Nat'l Acad. Sci. USA 84:7851 (1987); Biochemistry 28:908 (1989), which is hereby incorporated by reference). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

According to a preferred approach, the naked plasmid DNA or infective transformation vector can be injected directly into the bladder of the animal to be transformed during a laparotomy, and the animal subsequently overexpress the peptide in their bladder sensory neurons.

Identification of therapeutic agents can be achieved by providing suspected therapeutic agents to the transgenic animal overexpressing PACAP or CGRP or other neuropeptides, and then assessing the response of the animal following administration. Criteria to be used for evaluating efficacy include, without limitation, void volume, void frequency, void duration, flow rate, locomotor activity, body temperature, body weight, bladder permeability, sensitivity to KCl (Leung et al., Urology 64:378-382 (2004); Eichel et al., Urology 58:113-118 (2001); Wood et al., “Simultaneous evaluation of urothelial permeability, sensitivity, and voiding function in the mouse,” (abstract), Basic Research in Interstitial Cystitis, First Annual Investigators Meeting, Oct. 20, 2004 (Washington, D.C.), each of which is hereby incorporated by reference in its entirety).

### EXAMPLES

The following examples are intended to illustrate, but by no means are intended to limit, the scope of the present invention as set forth in the appended claims.

### Example 1

### Enzyme-Linked Immunosorbant Assay for CGRP

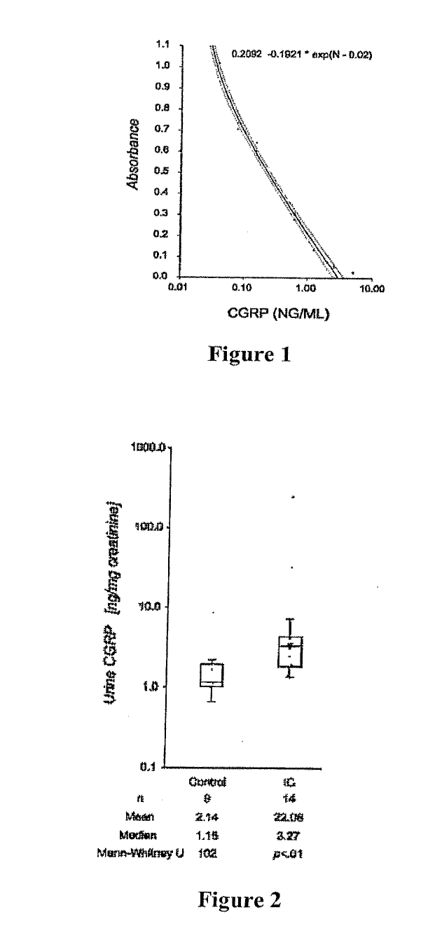

Urine samples were centrifuged at 2000×g for 5 min to pellet cellular debris, and then supernatants were aliquoted and stored at −70° C. prior to extraction and assay. Equal volumes of urine and 1% trifluoroacetic acid (TFA) were mixed, and then centrifuged at 6000×g for 20 minutes at 4° C. The resulting supernatant was collected for extraction. C18 Sep columns were equilibrated with 1 ml of 100% acetonitrile, and then washed three times with 3 ml 1% TFA prior to loading the prepared supernatant. After the clarified and acidified samples were loaded onto the column, the columns were once again washed with 1% TFA (3 ml, three times). Sample was eluted using 3 ml 60% acetonitrile in 1% TFA. Eluents were vacuum dried overnight, and then resuspended in the supplied assay buffer (Cat.# S-1199; Peninsula Laboratories, San Carlos, Calif.). CGRP concentrations were normalized against urine creatinine levels, as determined by colorimetric assay. Recovery experiments with iodinated CGRP indicated minimal adsorption of CGRP to the plastic tube. The calibration curve for the assay, bounded by 95% confidence intervals is shown in FIG. 1. A radioimmunoassay procedure is currently being implemented. Either method should be useful for the development of the mouse models, and for cell culture experiments.

### Example 2

### Demonstration of Elevated CGRP Excretion in Interstitial Cystitis

The ELISA according to Example 1 was used to provide a urine analysis for 9 controls and 15 patients. The medians for controls and IC patient groups were 1.21 and 3.11 ng/mg creatinine respectively. Note that 75% of the IC patients had CGRP levels greater than 1.92 ng/mg creatinine, and that 75% of control subjects had levels less than 1.82 ng/mg creatinine The concentration estimates were evaluated with a nonparametric test: Mann-Whitney U=105, p<0.01. A robust oneway analysis of variance was also significant: F_{(1, 21)}=6.85 p=0.0161. The demonstration of an elevation in CGRP in IC patients suggests that CGRP could have a role in the causation of or disposition to develop the disease, or may reflect a post-initiation process unique to this diagnosis. It may be causally or correlatively related to urgency, frequency, reduced bladder volume, or pain.

### Example 3

### Preparation of UPII-rtTA Transgene and Transgenic Mice

The UPII-rtTA transgene was constructed by replacing the lacZ reporter gene in the UPII-lacZ plasmid supplied by Dr. Sun (Mol. Biol. Rep. 23(1):3-11 (1996); U.S. Pat. No. 5,824,543 to Sun, each of which is hereby incorporated by reference in its entirety) with the rtTA cDNA from the pUHD172-1neo plasmid (now commercially available as Tet-On™ (BD Biosciences/Clontech). Transient transfection of MBT2 cells and induction of reporter genes with doxycycline confirmed biological activity of the transgene preparation before the investment of time and materials involved in generating transgenic mice.

The UPII-rtTA transgene was injected into 200 C57BL/6J x DBA2/J mouse zygotes and mice were screened for the transgene by polymerase chain reaction (PCR). Four founder mice identified. Germline transmission of the UPII-rtTA transgene has been obtained and a “best candidate” line has been bred to homozygosity. Expression of rtTA protein in bladder urothelium of this line has been confirmed by immunohistochemistry, with an antibody that recognizes an epitope in the carboxy terminus of the protein (FIG. 6).

### Example 4

### Successful Regulation of Gene Expression in Mouse Urothelium

The bi-transgenic system was first tested for expression of erb2 in mouse urothelial tissue. Four founder lines of UPII-rtTA mice were established as described in Example 3 above. These mice were crossed to a TRE-erbB2 line with the goal of achieving urothelium specific and doxycycline dependent expression of the erbB2 oncogene. While chronic over-expression of the erbB2 oncogene is expected to produce pre-malignant and malignant changes in the mouse urothelium, a series of short term experiments have been conducted on crosses between UPII-rtTA lines and the TRE-erbB2 line. These crosses produce four possible genotypes as outlined in Table 1 below.

TABLE 1
Genotype and Phenotype of Bi-Transgenic Erb2 Mice

| | | | | Number of animals |
|---|---|---|---|---|
| UPII- | UPII-rtTA | TRE-erbB2 | Number of | with sub-mucosal |
| rtTA line | genotype | genotype | Animals | inflammation |
| rtTA.72 | − | − | 1 | 0 |
| | + | − | 3 | 0 |
| | − | + | 5 | 0 |
| | + | + | 5 | 2 |
| rtTA.94 | − | − | 2 | 0 |
| | + | − | 0 | 0 |
| | − | + | 3 | 0 |
| | + | + | 3 | 2 |

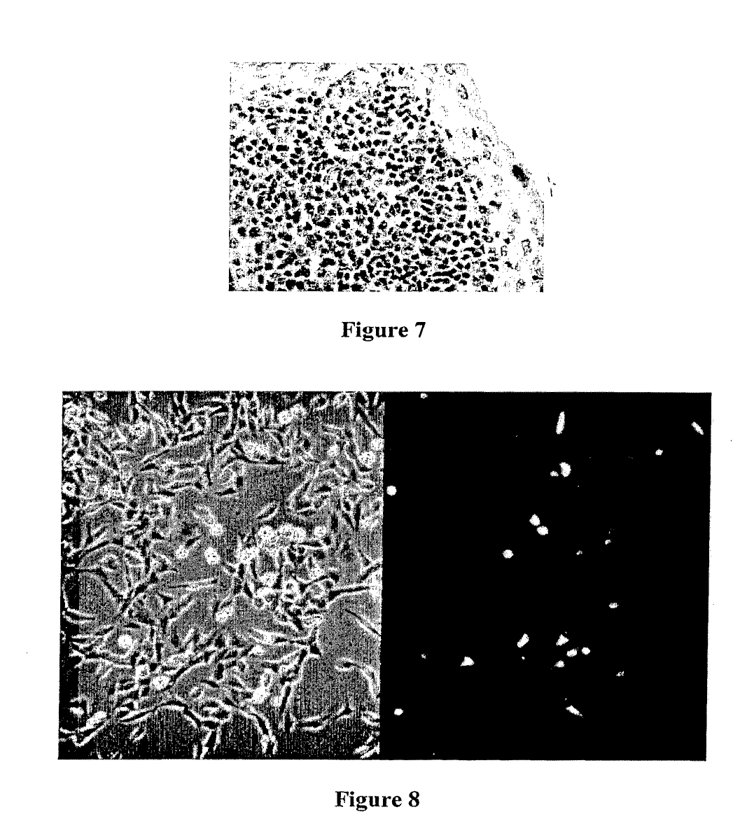

Beginning at ten weeks of age the mice were started on 1 mg/ml doxycycline ad libitum in their drinking water. Three weeks later they were euthanized and their bladders fixed in formalin then embedded in paraffin. Sections were hematoxylin and eosin stained. A distinct sub-mucosal inflammation (FIG. 7) was noted in bi-transgenic mice in two of the crosses (rtTA.72 and rtTa.94). These lesions were positive for expression of CD3, a T-cell marker. While this is a preliminary (and unexpected) result, the observation of the same phenotype in bi-transgenic mice from two independent lines and the absence of this phenotype in wildtype or mono-transgenic littermates supports the conclusion that the bi-transgenic strains exhibited regulated gene expression in the mouse urothelium.

### Example 5

### Preparation of PACAP/CGRP Genetic Constructs and Transgenic Mice

Both PACAP38 and CCRP are initially synthesized as propeptides (proPACAP and proCGRP, respectively) that undergo endoproteolytic cleavage and carboxy terminal amidation to become biologically active neuropeptides (Vaudry et al., Pharmacol. Rev. 52(2):269-324 (2000); Rosenblatt and Dickerson, Peptides 18(4): 567-576 (1997), each of which is hereby incorporated by reference in its entirety). To overcome the challenges of post-translational processing of proPACAP and proCGRP in urothelial cells, the carboxy terminal 38 amino acids of proPACAP and the carboxy terminal 37 amino acids of proCGRP will be cloned into the tetracycline responsive targeting vector, pTRE.

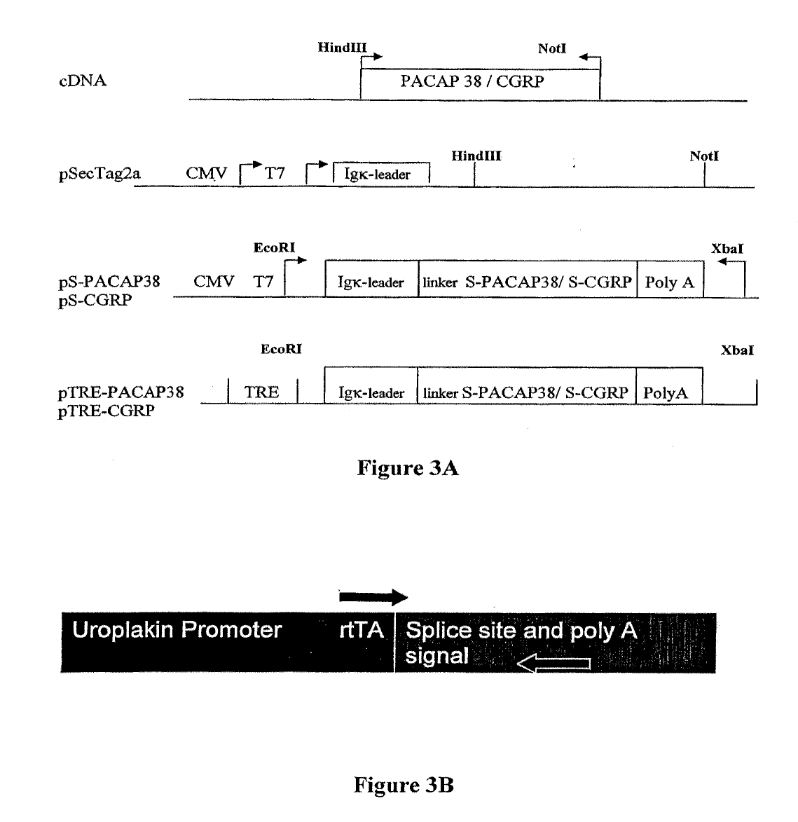

To ensure proper carboxy terminal amidation, peptidyl glycine α-amidating monooxygenase (PAM), will also be placed under control of the tetracycline response element in a separate pTRE construct (see, e.g., FIG. 3A). The resulting founder mice will be PAM/PACAP38 and PAM/CGRP. These bi-transgenic mice will be crossed with UPII-rtTa transgenic mice to make bi- and tri-transgenic strains.

### Generation of the TRE-PAM Targeting Vector:

To ensure proper post-translational amidation of the carboxy terminus of PACAP38, peptidyl glycine α-amidating monooxygenase (PAM), will also be cloned into a separate pTRE vector to generate a pTRE-PAM construct. The cDNA for PAM (Genbank Accession U79523, which is hereby incorporated by reference in its entirety) will be used as template for PCR with primers containing EcoRI (5′ CGCGAATTCACCATGGCCGGACGCGCCCGC, SEQ ID NO: 3) and XbaI (5′ GGCTCTAGATCAGGAGGAAGGTGCAGG, SEQ ID NO: 4) sites. The 2960 by product will then be ligated into linearized pTRE-Tight cloning vector (BD Biosciences/Clontech). Sequence analysis will confirm insertion of full length PAM.

### Fusing a Signal Sequence to the N-Terminus of PACAP38:

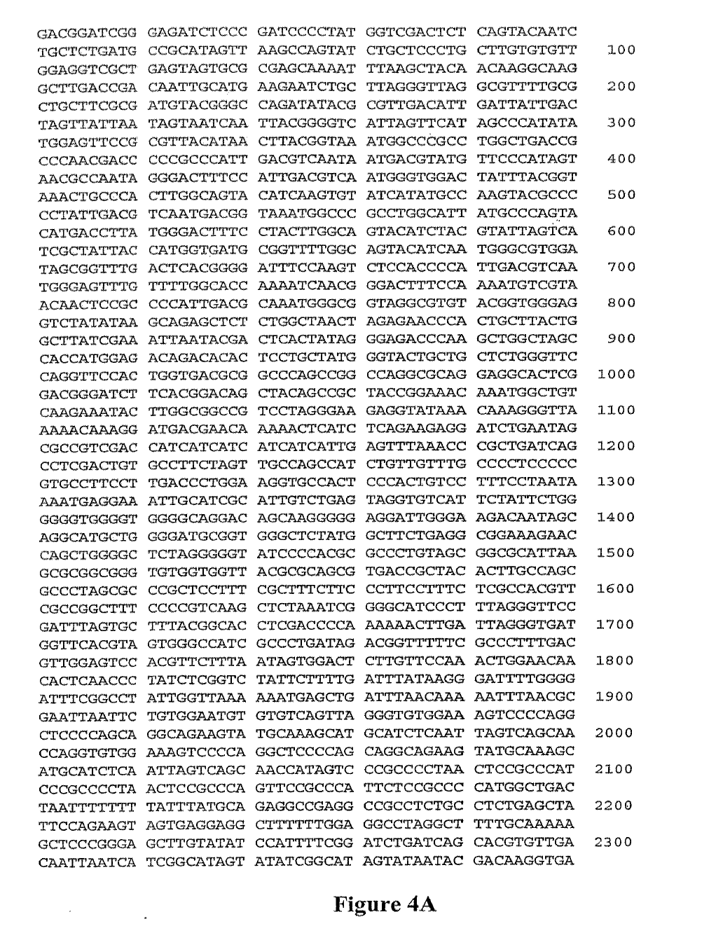

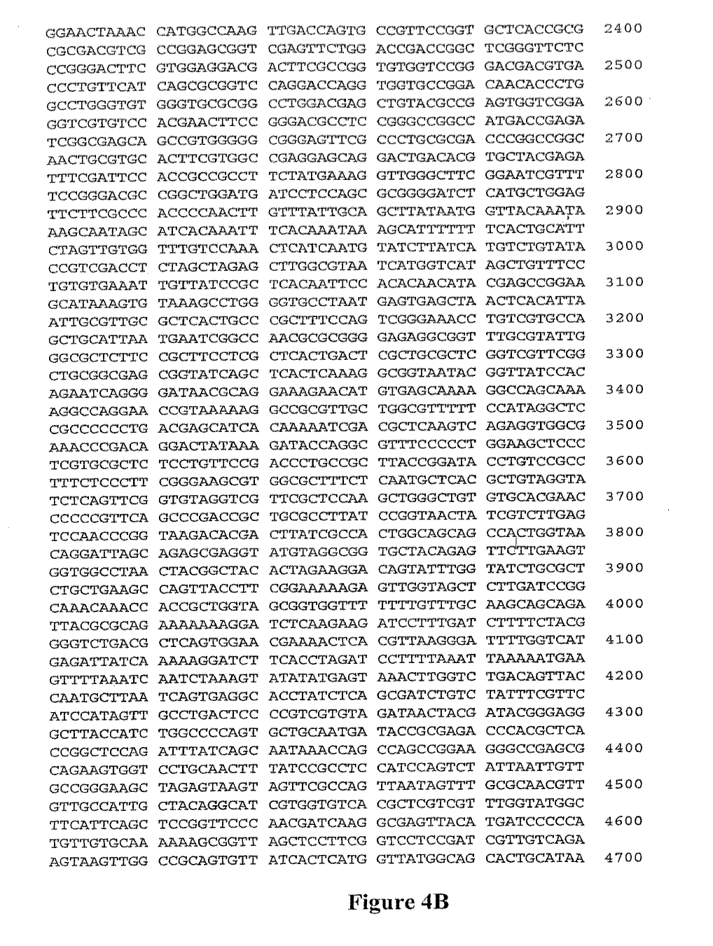

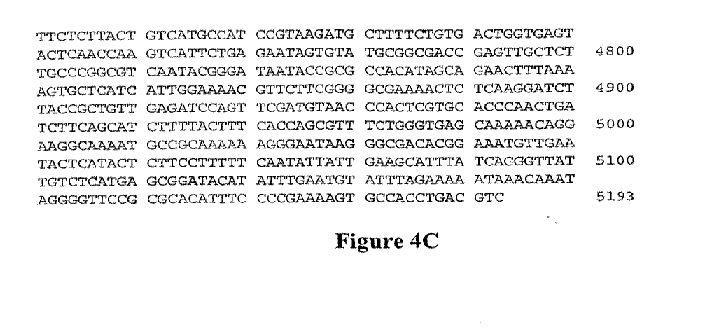

The PACAP38 peptide is made from proteolytic cleavage and processing of a larger pro-PACAP construct consisting of a total of 175 amino acids. The resulting 38 amino acid PACAP38 (corresponding to amino acids 131 to 169) also undergoes a post-translational amidation at the carboxy terminal end by the enzyme PAM. To generate the PACAP38 polypeptide, PCR using a high fidelity enzyme was performed with primers (5′ CGCAAGCTTTCACTCGGACGGGATCTTCACGGAC, SEQ ID NO: 5) with HindIII site and (5′ GCGGCGGCCGCTCATCCTTTGTTTTTAACCCT, SEQ ID NO: 6) with NotI site and an introduced stop site. Because the entire reading frame of PACAP38 is contained within exon 5, genomic DNA isolated from mouse liver was used as template. The 139 by product was ligated into linearized pSecTag2a vector in frame with Ig kappa leader sequence for efficient secretion. The entire fusion product, including the Ig kappa leader sequence, signal peptide cleavage site, and PACAP38 is 72 amino acids in length. The construct, called pS-PACAP38, has a nucleotide sequence as shown in FIGS. 4A-C.

### Generation of the TRE-PACAP38 Targeting Vector:

To clone the S-PACAP38 cDNA into the pTRE-Tight (BD Biosciences/Clontech), PCR amplification of the coding region will be performed with primers containing sites for EcoRI (5′ CGCGAATTCACCATGGAGACAGACACACTCC, SEQ ID NO: 7) and XbaI (5′ CGGCGTCTAGATCATCCTTTGTTTTTAACCC, SEQ ID NO: 8), or other suitable primers that can allow for integration into the multi-cloning site of pTRE-Tight empty vector. The resulting 241 by PCR product will be ligated into the linearized pTRE-Tight vector, forming pTRE-PACAP38. The resulting construct will be analyzed to confirm correct fusion by sequencing both strands of the insert and the junction. This targeting vector will then be transfected into the HeLa-TetON line, which is stably transformed with rtTA, to confirm the doxycycline dependent expression of S-PACAP38 by ELISA and radio immune assays.

### Fusing a Signal Sequence to the N-Terminus of CGRP:

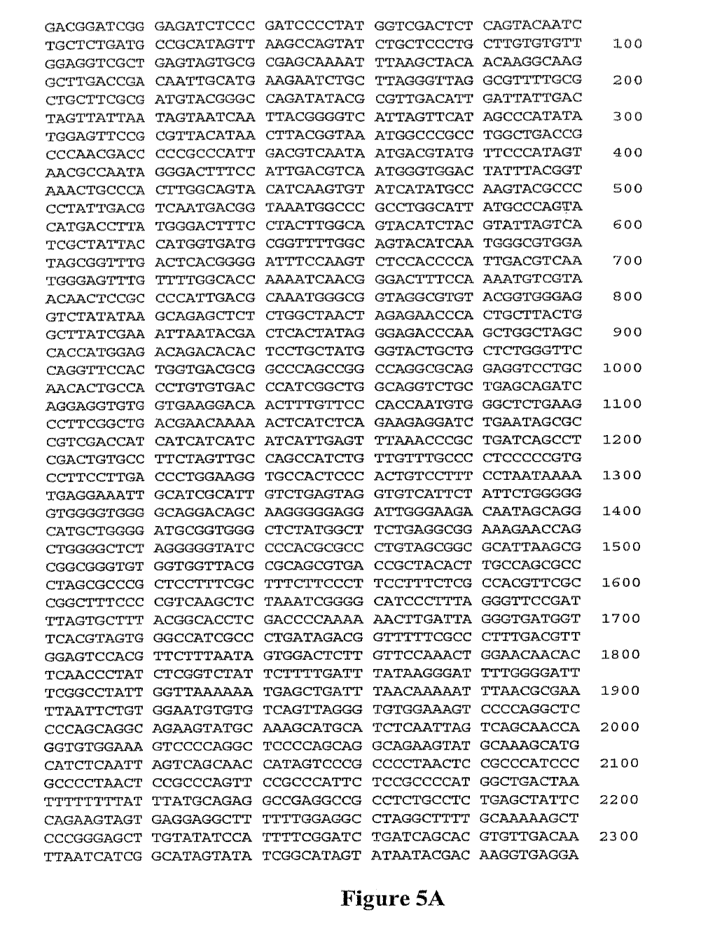

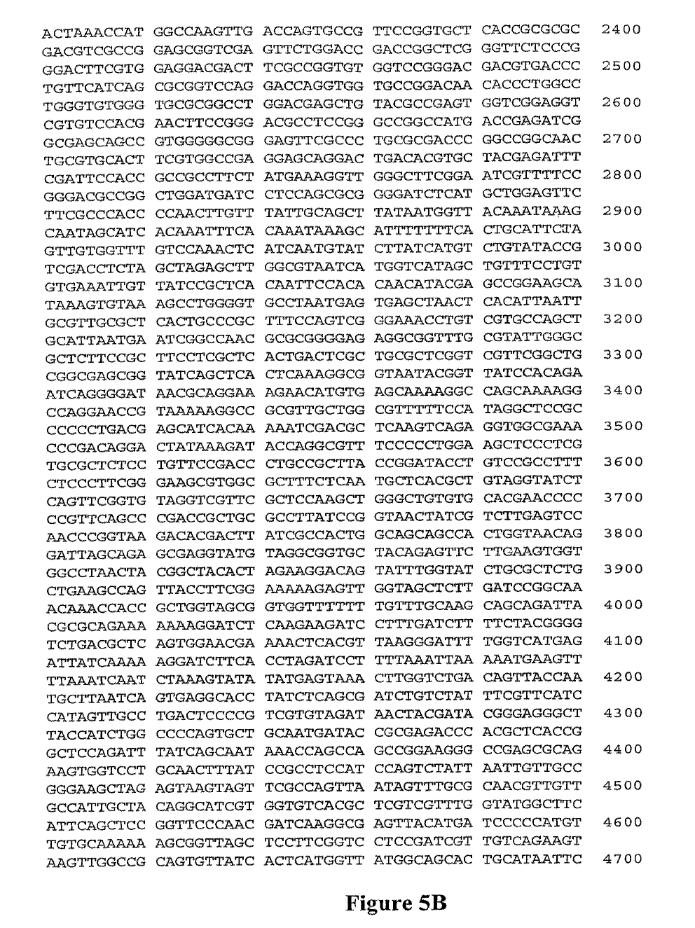

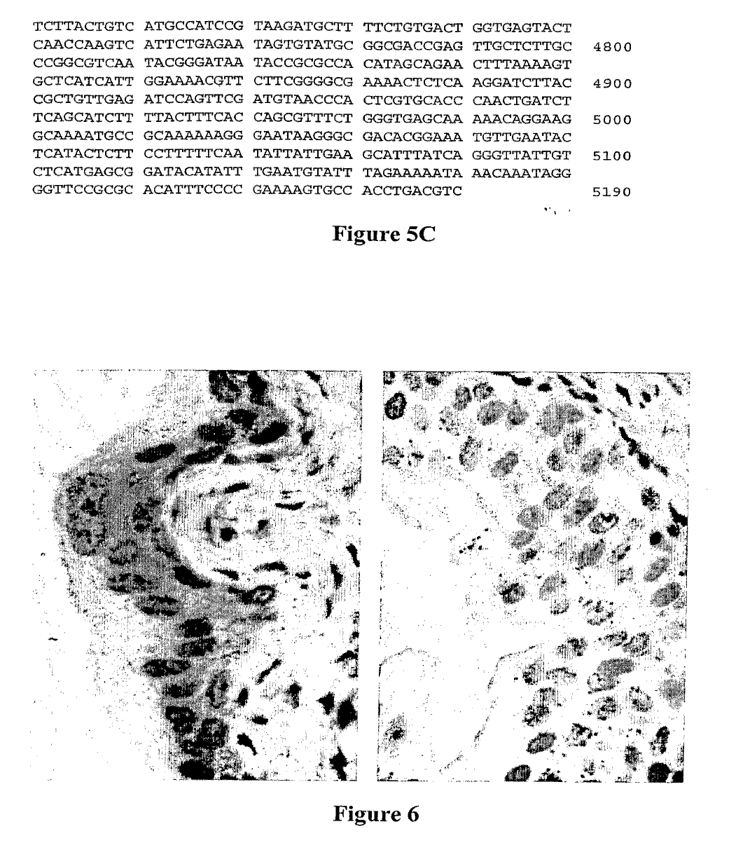

The CGRP peptide is made from proteolytic cleavage and processing of a larger pro-CGRP construct consisting of a total of 38 amino acids (corresponding to amino acids 83 to 120). CGRP also undergoes a post-translational amidation at the carboxy terminal end by the enzyme PAM. First strand reverse transcription was performed using RNA purified from mouse brain as template to generate cDNA for second round PCR. To then generate the CGRP polypeptide, PCR using a high fidelity enzyme was performed with primers (5′ CGCAAGCTTTTCCTGCAACACTGCCACCTG, SEQ ID NO: 9) with a HindIII site and (5′GTGGGCTCTGAAGCCTTCGGCTGAGCGGCCGCCGC, SEQ ID NO: 10) with a NotI site and an introduced stop site. The 140 by product was ligated into linearized pSecTag2a vector in frame with Ig kappa leader sequence for efficient secretion. The entire fusion product, including the Ig kappa leader sequence, signal peptide cleavage site, and CGRP is 71 amino acids in length. The construct, called pS-CGRP, has a nucleotide sequence as shown in FIGS. 5A-C.

### Generation of the TRE-CGRP Targeting Vector:

To clone the S-CGRP cDNA into the pTRE-Tight (BD Biosciences/Clontech), PCR amplification of the coding region will be performed with primers containing sites for EcoRI (5′ CGCGAATTCACCATGGAGACAGACACAC, SEQ ID NO: 11) and XbaI (5′ GTGGGCTCTGAAGCCTTCGGCTCTAGACGC, SEQ ID NO: 12), or other suitable primers that can allow for integration into the multi-cloning site of pTRE-Tight empty vector. The resulting 236 by PCR product will be ligated into the linearized pTRE-Tight vector, forming pTRE-CGRP. The resulting construct will be analyzed to confirm correct fusion by sequencing both strands of the insert and the junction. This targeting vector will then be transfected into the HeLa-TetON line, which is stably transformed with rtTA, to confirm the doxycycline dependent expression of S-CGRP by ELISA and radio immune assays.

Generation of TRE-PACAP38 Transgenic Mice:

The fusion gene will be excised, gel purified, and then microinjected into 200 fertilized mouse eggs (C57BL/6J X DBA2). These eggs will be implanted into CD-1 foster mothers. The S-PACAP38 transgene will be identified by PCR and Southern blot analysis (using a 500 bp probe from the TRE promoter region) of tail DNA. Positive founder animals will be backcrossed with (C57BL/6JXDBA2) F1 hybrids to generate hemizygous animals that will be used to study transgene expression. Up to three founder lines will be established and analyzed for constitutive PACAP38 expression in their urine by Western blot and urothelium by immunohistochemistry.

### Generation of TRE-PAM Transgenic Mice:

The fusion gene will be excised, gel purified, and then microinjected into 200 fertilized mouse eggs (C57BL/6J X DBA2). These eggs will be implanted into CD-1 foster mothers. The PAM transgene will be identified by PCR and Southern blot analysis (using a 500 bp probe from the TRE promoter region) of tail DNA. Positive founder animals will be backcrossed with (C57BL/6JXDBA2) F1 hybrids to generate hemizygous animals that will be used to study transgene expression. Up to three founder lines will be established and analyzed for constitutive PAM expression in their urine by Western blot and urothelium by immunohistochemistry as described earlier.

### Generation of TRE-CGRP Transgenic Mice:

The fusion gene will be excised, gel purified, and then microinjected into 200 fertilized mouse eggs (C57BL/6J X DBA2). These eggs will be implanted into CD-1 foster mothers. The S-PACAP38 transgene will be identified by PCR and Southern blot analysis (using a 500 bp probe from the TRE promoter region) of tail DNA. Positive founder animals will be backcrossed with (C57BL/6JXDBA2) F1 hybrids to generate hemizygous animals that will be used to study transgene expression. Up to three founder lines will be established and analyzed for constitutive CGRP expression in their urine by Western blot and urothelium by immunohistochemistry as described earlier.

### Generation and Analysis of the Uro-TetON-PACAP38/CGRP Transgenic Mice:

The TRE-PACAP38 transgenic mice that have little or no background urothelial expression will be crossed with UPII-rtTA transgenic mice to make the double transgenics. These Uro-TetON-PACAP38 transgenic mice will be confirmed by standard genotyping procedure from tail DNA. Up to three founder lines will be established and analyzed for PACAP38 expression and voiding function. Twenty-four mice from each founder line will be divided into 4 groups of 6 age-(>8 wk.) and sex-matched mice. Baseline micturition frequency and volume for each mouse will be obtained over 2-weeks. Baseline urine samples at 0, 1 and 2 weeks will be stored at −80° C. Then the mice will be given doxycycline for 2 weeks: Group 1 (control, 0 μg/ml), Group 2 (50 μg/ml), Group 3 (100 μg/ml) and Group 4 (200 μg/ml). (Subsequent experiments will use a modified Latin-square design in which dose-effect functions are derived in individual animals.) Urine samples will be collected daily, tested for hematuria (Wood et al., Urology 57(6 Suppl 1):115-116 (2001), which is hereby incorporated by reference in its entirety), and stored at −80° C. Euthanized after a month of functional observation, bladder, kidney, spinal cord, liver, lung, heart and brain samples will be harvested. The spinal cord and half of the bladder will be processed for immunocytochemistry. Total RNA will be prepared from the other half of the bladder and the remaining tissues, and PACAP38 mRNA expression will be determined by real time RT-PCR as previously described (Zhang et al., J. Clin. Invest. 109(11):1405-1415 (2002), which is hereby incorporated by reference in its entirety). Urine samples will be analyzed for PACAP38 by ELISA using creatinine and total protein for standardization. Uro-TetON-CGRP mice will be generated similarly.

### Example 6

### Generation of Neuropeptide-Overexpressing Somatic Mosaic Mice

The cDNA encoding the secreted PACAP38 or CGRP will be subcloned into a replication defective recombinant feline immunodeficiency virus (FIV) transfer vector (Kyrkanides et al., Mol. Brain Res. 119:1-9 (2003), which is hereby incorporated by reference in its entirety), where it will be under the transcriptional control of the neuron specific enolase (NSE) promoter. This plasmid will be used to make recombinant retrovirus that will be injected into the bladder wall of adult female mice during laparotomy. These PACAP38 or CGRP somatic mosaic mice will be analyzed for PACAP38 or CGRP expression in their sensory neurons and alterations in micturition function using FIV-NSE-lacZ infected mice as controls.

### Generating the FIV-NSE-(Neuropeptide) Retroviral Vector:

The FIV-NSE-(PACAP38 or CGRP) transfer vector will be made by replacing the β-galactosidase gene in pFIV-NSE-lacZ (Kyrkanides et al., Mol. Brain Res. 119:1-9 (2003), which is hereby incorporated by reference in its entirety) with the open reading frame of pS-PACAP38 (or pS-CGRP), via direct subcloning into the EcoRI/XbaI restriction sites. This vector will be co-transfected with the pFIV helper plasmid and pVSV-G pseudotyping plasmid into 293T cells and the recombinant viral vector will be purified and concentrated to titers of 10⁷-10⁸/ml. The tissue-restricted neuropeptide expression will be examined in vitro by infecting 293T cells and NGF-differentiated PC12 cells and analyzing the media for neuropeptide levels by ELISA at 0, 12, 24, 48 and 72 hrs post-infection.

### Generation and Analysis of Neuropeptide Somatic Mosaic Mice:

Twenty-four, 8-week-old female C57B16 mice will be divided into 4 groups. The mice will be placed in the metabolism cages and baseline data on micturition frequency and volume will be obtained over 2-weeks. Baseline urine samples at 0, 1 and 2 weeks will also be obtained and stored at −80° C. Then an open laparotomy will be performed under deep anesthesia (2% halothane) to expose the bladder wall. The mice will be given five direct injections into their bladder wall, 10 μl each and 50 μl total volume in the following groups: Group 1 (control, 10⁶ infectious particles of FIV-lacZ),

Group 2 (10⁵ infectious particles of FIV-neuropeptide), Group 3 (5×10⁵ infectious particles of FIV-neuropeptide) and Group 4 (10⁶ infectious particles of FIV-neuropeptide). The mice will be placed in the metabolism cages for 2 more weeks, and urine samples collected daily, tested for hematuria (Wood et al., Urology 57(6 Suppl 1):115-116 (2001), which is hereby incorporated by reference in its entirety), and stored at −80° C. After a month of observation, tissues will be harvested as described above. The spinal cord and half of the bladder will be processed for neuropeptide immunohistochemistry. Total RNA will be prepared from the other half of the bladder and the remaining tissues, and neuropeptide mRNA expression will be determined by real time RT-PCR as previously described (Zhang et al., J. Clin. Invest. 109(11):1405-1415 (2002), which is hereby incorporated by reference in its entirety). The urine samples will be analyzed for neuropeptide by ELISA using creatinine and total protein for standardization.

### Example 7

### In Vitro Analysis of Transgenic Cells Expressing Secretable CGRP or PACAP

Before completing preparation of the pTRE-CGRP or pTRE-PACAP38 plasmids (see FIG. 3A and Example 2 above), the pS-CGRP and pS-PACAP38 secretion constructs were first tested to assess secretion of the polypeptides. The secretion constructs were prepared as described in Example 5 and as illustrated in FIG. 3A, using the Invitrogen pSecTag2a plasmid. (The CMV promoter driven open reading frame starts at nucleotide 905 in the sequences below. They carry the ampicillin resistance gene.)

pS-CGRP and pS-PACAP38 have been validated by sequencing in both the sense and anti-sense directions. The open reading frames of these constructs are as follows:

IgK secretion—(PARR cleavage)—EGFP

IgK secretion—(PARRRR cleavage)—CGRP

IgK secretion—(PARRRR cleavage)—PACAP

The complete nucleotide sequence of pS-PACAP38 appears in FIGS. 4A-C, and the complete nucleotide sequence of pS-CGRP appears in FIGS. 5A-C. In addition to pS-CGRP and pS-PACAP38, pS-EGFP was prepared as a control.

UMUC3 cells were co-transfected in a six well plate with 1 ug of each of the EGFP, CGRP, and PACAP plasmids together with 1 ug of CRE-Luc reporter plasmid in Lipofectamine. The EGFP fluorescence allowed for visualization of transfection efficiency. FIG. 8 (left panel) is a phase constrast image of the pS-EGFP/pCRE-Luc transfected cells taken two days after addition of the plasmids to the culture media. FIG. 8 (right panel) is the green fluorescence image of the same field.

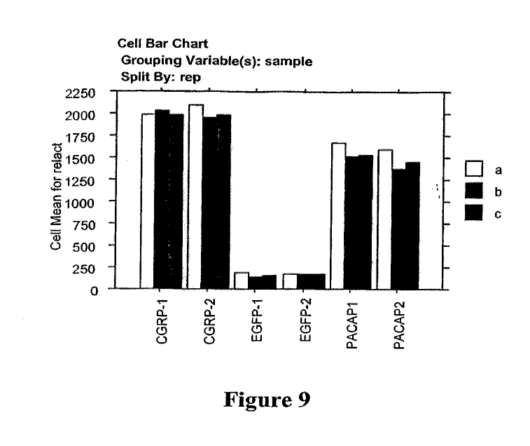

After imaging, the media was removed and centrifuged to remove large particulates and then frozen at −85° C. for assay of CGRP and PACAP. The cells were harvested by scraping and centrifuged to pellet. They were lysed in 100 ul of Promega Reporter Lysis Buffer. Total protein yield for each well was determined by Bradford assay. Luciferase activity was assayed by measurement of luminescence after addition of 20 ul of cell lysate to 100 ul of Reporter Lysis Reagent. The results for three replicate measurements of luciferase activity for duplicate wells are shown in FIG. 9, which demonstrates that the CGRP and PACAP secretion constructs are triggering CRE-Luc activity in UMUC3 cells, implying the production of functional CGRP and PACAP peptides. In addition, it appears that the UMUC3 cell line has the capacity to respond to both peptides.

The CGRP and PACAP-expressing open reading frames can now be cloned into the TRE-Tight vector or FIV-vector to allow for preparation of transgenic mice expressing either CGRP or PACAP, either as a bi- or tri-transgenic or as a somatic mosaic in accordance with the present invention.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of diagnosing interstitial cystitis comprising:
measuring a level of CGRP in a sample obtained from a patient having a symptom of interstitial cystitis; and
determining if the measured level of CGRP in the sample is elevated in relation to a standard level of CGRP in a normal asymptomatic population, wherein the measured level of CGRP that is elevated relative to the standard level in combination with the symptom indicates the diagnosis of a interstitial cystitis.

2. The method according to claim 1, wherein said measuring comprises use of CGRP-specific antibodies.

3. The method according to claim 1, wherein said measuring comprises use of HPLC, mass spectrometry, or an assay system selected from the group of enzyme-linked immunoabsorbent assay, radioimmunoassay, gel diffusion precipitin reaction assay, immunodiffusion assay, agglutination assay, fluorescent immunoassay, protein A immunoassay, and immunoelectrophoresis assay.

4. The method according to claim 1, wherein the sample is a urine sample, a blood sample, or a spinal fluid sample.

5. The method according to claim 1, wherein the patient is a human.

6. The method according to claim 1 wherein the symptom comprises one or more of pain during urination, urgency of urination, frequency of urination, ulcers of bladder mucosa, and petechial hemorrhages of bladder mucosa.

7. The method according to claim 1 further comprising treating the patient for the diagnosed interstitial cystitis.

8. A method of diagnosing a pelvic pain disorder comprising:
measuring a level of CGRP in a sample obtained from a patient having a symptom of a pelvic pain disorder; and
determining if the measured level of CGRP in the sample is elevated in relation to a standard level of CGRP in a normal asymptomatic population, wherein the measured level of CGRP that is elevated relative to the standard level in combination with the symptom indicates the diagnosis of a pelvic pain disorder.

9. The method according to claim 8, wherein said measuring comprises use of CGRP-specific antibodies in an assay selected from the group of enzyme-linked immunoabsorbent assay, radioimmunoassay, gel diffusion precipitin reaction assay, immunodiffusion assay, agglutination assay, fluorescent immunoassay, protein A immunoassay, and immunoelectrophoresis assay.

10. The method according to claim 8, wherein the sample is a urine sample, a blood sample, or a spinal fluid sample.

11. The method according to claim 8, wherein the pelvic pain disorder is interstitial cystitis, Crohn's disease, ulcerative colitis, irritable bowel syndrome, vulvodynia, vestibulitis, endometriosis, prostatitis, orchalgia, or proctalgia.

12. The method according to claim 8 further comprising treating the patient for the diagnosed pelvic pain disorder.

13. A method of treating a pelvic pain disorder in a patient comprising providing a CGRP antagonist and administering the CGRP antagonist to a patient in an amount effective to treat the pelvic pain disorder.

14. The method according to claim 13, wherein the CGRP antagonist is BIBN4096BS, SB-(+)-273779 [N-methyl-N-(2-methylphenyl)-3-nitro-4-(2-thiazolylsulfinyl)nitrobenzanilide], or a fragment of CGRP.

15. The method according to claim 13, wherein said administering is carried out orally, parenterally, subcutaneously, transdermally, intravenously, intramuscularly, intraperitoneally, by intranasal instillation, by implantation, by intracavitary or intravesical instillation, intraocularly, intraarterially, intralesionally, by application to mucous membranes, or by intrabladder administration.

16. The method according to claim 13, wherein the CGRP antagonist is present in a pharmaceutical composition comprising the CGRP antagonist and a pharmaceutically-acceptable carrier.

17. The method according to claim 13, wherein the patient is a human.

18. The method according to claim 13, wherein said administering is effective to mitigate symptoms of the pelvic pain disorder.

19. The method according to claim 18, wherein the symptoms of the pelvic pain disorder comprise one or more of pain during urination, urgency of urination, frequency of urination, ulcers of bladder mucosa, and petechial hemorrhages of bladder mucosa.

20. The method according to claim 13, wherein the pelvic pain disorder is interstitial cystitis, Crohn's disease, ulcerative colitis, irritable bowel syndrome, vulvodynia, vestibulitis, endometriosis, prostatitis, orchalgia, or proctalgia.
